(19) United States
(12) Patent Application Publication
(10) Pub. No.: US 2004/0156904 A1
(43) Pub. Date: Aug. 12, 2004

(54) Biodegradable polymer device

(75) Inventors: Adam Saltman, Westborough, MA (US); Glenn Gaudette, E. Setauket, NY (US); Weiliam Chen, Mount Sinai, NY (US); Hongliang Jiang, South Setauket, NY (US); Yang Hyun Yun, Huntington, NY (US)

Correspondence Address: PITNEY HARDIN LLP, 7 TIMES SQUARE, NEW YORK, NY, 10036-7311, US

(73) Assignee: The Research Foundation of State University of New York

(21) Appl. No.: 10/364,877

(22) Filed: Feb. 12, 2003

(51) Int. Cl. A61K 48/00; A61K 9/14

(52) U.S. Cl. 424/486; 514/44; 514/55

(57) ABSTRACT

This invention provides a composition and method for preparing a biomedical device capable of delivering pharmaceutical or biomedical materials from a PEG-g-chitosan matrix. By combining a PEG-g-chitosan and a water insoluble polymer in a nonaqueous solvent, a matrix is obtained which can be used as a delivery vehicle for pharmaceuticals and biomedical materials.

This invention relates to a composition and method for preparing a biomedical device capable of delivering pharmaceutical or biomedical materials from a PEG-g-chitosan matrix. By combining a PEG-g-chitosan and a water insoluble polymer in a nonaqueous solvent, a matrix is obtained which can be used as a delivery vehicle for pharmaceuticals and biomedical materials. A biomedical device according to the invention including an anti-inflammatory agent in such a matrix has been found to reduce the incidence of post operative atrial fibrillation. This invention was supported by an Innovative Development and BIOCAT grant 2-8521 from the New York Office of Science, Technology and Academic Research which may have rights in the invention.

### BACKGROUND OF THE INVENTION

Drugs can be incorporated directly into the drug delivery matrices including chitosan, however drug release behavior is generally governed by polymer degradation as well as the morphologies of the dosage forms. Initial burst releases are frequently observed when hydrophilic drugs are used and fine modulation of drug release kinetics is challenging. In addition, bioactive agents such as proteins and DNA may lose their bioactivities when exposed to the hydrophobic surfaces and degradation products of polymers.

Since hydrophobic polymers lack functional groups, it is difficult to conjugate targeting moieties or specific ligands to render them targetable or bio-specific. This in turn limits their applications in drug delivery and tissue engineering. For water-soluble polymers, drugs could either be directly entrapped in the polymeric hydrogels or conjugated to their side chain to form pendant drugs. However, water-soluble polymers in general are difficult to process, and hydrogels prepared by crosslinking water-soluble polymers have low mechanical strengths, especially in the swollen states. In addition, most crosslinking agents used to prepare hydrogels have potential toxicity. Combining hydrophobic and water-soluble polymers could theoretically circumvent the shortcomings of the individual materials; however, it is technically difficult to blend the two types of polymers in a one-step process. In addition, water-soluble polymers tend to leach out rapidly from the blends when incubated in an aqueous environment.

Chitin is a naturally occurring polymer present in the fungi and the exoskeletons of crustaceans and insects. Chitosan is formed from chitin upon deacetylation of chitin by treatment with strong base. Chitosan, is typically 80-90% deacetylated as compared to chitin and is soluble in aqueous acid but is insoluble in water and nonaqueous solvents. Despite its lack of solubility and its brittleness, there has been significant effort expended in using chitosan as a drug delivery system because it is biocompatible and bioadhesive. Unlike many biodegradable polymers which induce inflammatory response, chitosan is non-inflammatory.

Chitosan can be modified by covalently bonding a poly(ethylene glycol) moiety through the amino function, sometimes referred to as PEGylation to form PEG-g-chitosan. Unfortunately, it is difficult to process PEG-g-chitosan, per se, to form drug delivery systems. Further, PEG-g-chitosan is a brittle material and the time release profile of drug delivery from systems made from PEG-g-chitosan is often unpredictable.

### SUMMARY OF THE INVENTION

It is an object of the invention to provide a time release pharmaceutical or biomedical delivery system including PEG-g-chitosan which is easy to process and delivers pharmaceuticals and biomedical materials reliably and consistently.

Another object of the invention is to provide a method for delivering pharmaceutical and biomedical materials to a tissue over a fixed time period in a reliable and predictable manner.

A further object of the invention is to provide a method for reducing inflammation and arrhythmogenesis of cardiac tissue.

These and other objects of the invention are achieved by providing a composition including PEG-g-chitosan and a water insoluble polymer in a nonaqueous solvent. Upon evaporation of the solvent, a biocompatible bioadhesive matrix is obtained which can be used as a delivery system. A pharmaceutical or biomedical material can be incorporated into the composition prior to evaporation or impregnated after formation in the biocompatible bioadhesive matrix. A pharmaceutical delivery system incorporating an anti-inflammatory pharmaceutical such as ibuprofen can significantly reduce inflammation of cardiac tissue thereby reducing the incidence of atrial fibrillation and cardiac arrhythmia.

### BRIEF DESCRIPTION OF THE DRAWING

FIG. 1 is a representative formula for PEG-g-chitosan;

FIGS. 2(a) and 2(b) are photographs of 90:10 PLGA:PEG-g-chitosan membrane and 70:30 PLGA:PEG-g-chitosan membrane, respectively after exposure to water;

FIG. 3 is a schematic illustration of the electrospinning process;

FIG. 4 is an illustration of a method of making PEG-g-chitosan;

FIG. 5 is an illustration of a method for coupling ibuprofen to PEG-g-chitosan;

FIG. 6 is an NMR spectrum for ibuprofen coupled to PEG-g-chitosan;

FIG. 7 is an electrospinning arrangement;

FIGS. 8 (a) and 8(b) are schematic illustrations of a fiber mesh and micro/nanoparticles embedded in fiber mesh;

FIG. 9(a)-(f) are scanning electron micrographs of various electrospun membrane preparations; having PLA:PEG-g-chitosan ratios of (a) 90:10, (b) 80:20, (c) 90:10, (d) 70:30, (e) 80:20, (f) 60:40;

FIG. 10. is a schematic illustration of a PEG-g-chitosan/PLGA membrane including ibuprofen overlaying atrial tissue;

FIG. 11 is a graph of ibuprofen release (0.9%) over time from a PEG-g-chitosan/PLGA film;

FIG. 12 is a graph of ibuprofen release from (i) pure PLGA film with 5% ibuprofen, (ii) PLGA/PEG-g-chitosan (70:30 ratio) films with 5% ibuprofen and (iii) PLGA/PEG-g-chitosan with 4.7% ibuprofen covalently conjugated; and

FIG. 13A-13D are photographs of excised rat hindlimbs stained with X-gal reagent.

### DETAILED DESCRIPTION OF THE PREFERRED EMBODIMENTS

This invention provides for a time release pharmaceutical or biomedical delivery system in which a pharmaceutical or biomedical agent is incorporated in a matrix prepared from a composition including PEG-g-chitosan and a water insoluble polymer in a nonaqueous solvent.

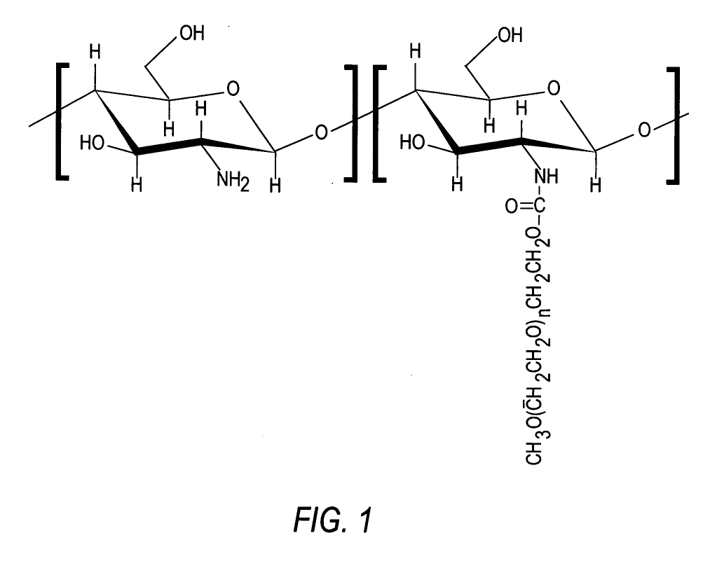

A representative formula for PEG-g-chitosan is illustrated in FIG. 1. Surprisingly, while the use of the water insoluble polymer alone results in delivery systems which are difficult to process and exhibit shrinkage after water exposure, the combination of the water insoluble polymer with PEG-g-chitosan in nonaqueous solvent results in a delivery system which is strong, not brittle, highly biocompatible, does not elicit an inflammatory response, and reliably delivers the biomedical or pharmaceutical agent over a fixed time period. Even more surprisingly, no crosslinking or covalent bonding of the PEG-g-chitosan and water insoluble polymer is required to achieve this synergistic result.

Suitable water insoluble polymers include but are not limited to a water insoluble polymer selected from the group consisting of polylactic acid, poly(lactide-co-glycolide) (PLGA), polycaprolactones, ethylene vinyl acetate, polyanhydride and mixtures thereof. Preferably, the water insoluble polymer is not polyethylene or polypropylene. Suitable non aqueous solvents include but are not limited to dimethylformamide (DMF), dimethylsulfoxide (DMSO), chloroform and mixtures thereof.

For example, blending of PEG-g-Chitosan and PLGA results in a composite material that has the desired properties, but not the disadvantageous properties of either component. The properties of the individual polymers are summarized in Table 1 of the individual polymers.

TABLE 1

| | PLGA | PEG-g-Chitosan |
|---|---|---|
| Mechanical Strength | Strong | Brittle/Poor |
| Functional Group | Lack | Abundant |
| Biocompatibilty | Medium | High |
| Incorporation of | Require relatively harsh | By simple |
| Bioactive Agents | conditions that may | chelation |
| (Proteins, DNA, | destroy the functions of | Stabilization |
| etc.) | the bioactive agents | |
| Drug Release | Hydrophilic drug - | Vary |
| Behavior | rapid burst | |
| | Hydrophobic drug - | |
| | sustained release | |
| Inflammatory Response | Yes | No |

PLGA can be used to prepare delivery systems from electrospun membranes. However, these membrane can shrink by more than 80% after immersion in distilled water at 37° C. for up to 2 hours. In contrast electrospun membranes from PEG-g-chitosan and PLGA are resistant to shrinkage after water exposure.

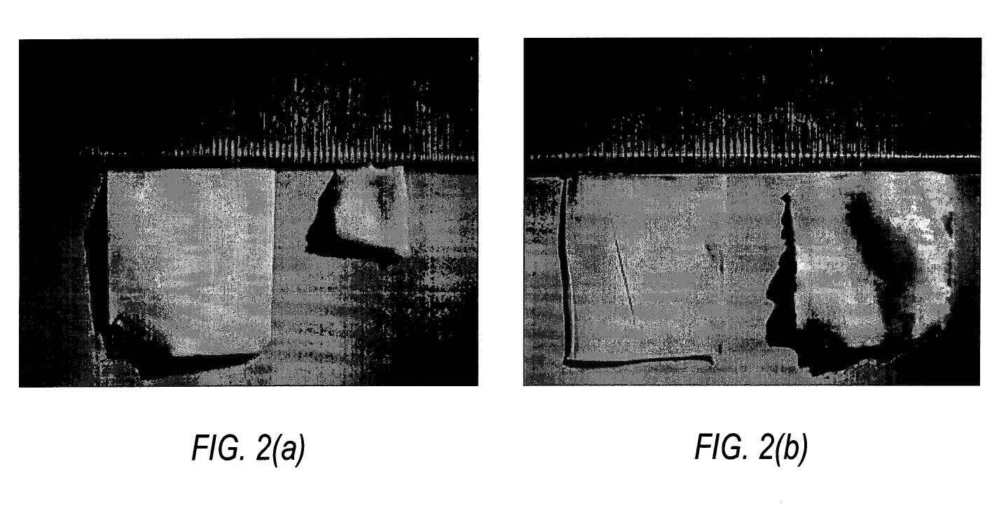

The shrinkage of PLGA/PEG-g-chitosan films decreased with an increase in PEG-g-chitosan content in the blend. A photograph of PLGA:PEG-g-chitosan (90:10) (by weight) after exposure to water is shown in FIG. 2(a) and a PLGA:PEG-g-chitosan (70:30) by weight membrane is shown in FIG. 2(b).The film prepared by PLGA/PEG-g-chitosan at a 90/10 ratio contracted to 24% of its original size when the film was immersed in distilled water at 37° C. However, the extent of shrinkage was drastically reduced with an increase in the film PEG-g-chitosan content, only 3% shrinkage was observed for the film prepared by PLGA/PEG-g-chitosan at a 70/30 ratio. The results may be attributed to both the less porous structure of the films with higher PEG-g-chitosan content and the hydrophilicity of PEG-g-chitosan, which tend to swell when exposed to water.

Any pharmaceutical or biomedical agent can be included in the delivery system. By “pharmaceutical or biomedical agent” is meant a biologically active molecule that can be used in the treatment, cure, prevention or diagnosis of disease or is otherwise used to enhance physical or mental well being in humans or other animals. Suitable pharmaceutical agents include but are not limited to analgesics such as acetaminophen, anti-inflammatory agents, antimicrobials, antivirals, antifungals, antiarrythmics and antitumor agents.

Antimicrobials that may be used in accordance with the present invention include all antibiotics, antimicrobial agents and antimicrobial peptides. Antibiotics that may be used include dermatologically acceptable salts of tetracyline and tetracycline derivatives, gentamycin, kanamycin, streptomycin, neomycin, capreomycin, lineomycin, paromomycin, tobramycin, erythromycin, triclosan, octopirox, parachlorometa xylenol nystatin, tolnafiate, miconazole hydrochloride, chlorhexidine gluconate, chlorhexidin hydrochloride, methanamine hippurate, methanamine mandelate, minocycline hydrochloride, clindamycin, cloecin, b-lactam derivatives such as aminopenicillin, chlorhexidin gluconate, and tricolosan and mixtures thereof.

Anti-inflammatory actives useful in accordance with the present invention include steroidal actives such as hydrocortisone as well as non-steroidal actives including propioinic derivatives; acetic acid derivatives; biphenylcarboxylic acid derivatives, fenamic acid derivatives; and oxicams. Example of anti-inflammatory actives include without limitation ibuprofen, acetosalicylic acid, oxaprozin, pranoprofen, benoxaprofen, bucloxic acid, elocon; and mixtures thereof.

Vitamin actives which may be used in accordance with the present invention include vitamin A and derivatives, including retonic acid, retinyl aldehyde, retin A, retinyl palimate, adapalene, beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-toco-pherol and cotrienols and mixtures thereof and vitamin E derivatives including vitamine E palmitate, vitamin E linolate and vitamin E acetate; vitamin K and derivatives; vitamin Q (ubiquinone) and mixtures.

Antiarrhythmics can be incorporated into the delivery system. Antiarrhythmics which may be used in accordance with the present invention include Acebutolol, Acecainide, Adenosine, Ajmaline, Alprenolol, Amiodarone, Aprindine, Arotinolol, Atenolol, Azimilide, Bevantolol, Bidisomide, Bretylium Tosylate, Bucumolol, Bufetolol, Bunaftine, Bunitrolol, Bupranolol, Butidrine Hydrochloride, Butobendine, Capobenic Acid, Carazolol, Carteolol, Cifenline, Cloranolol, Disopyramide, Dofetilide, Encainide, Esmolol, Flecainide, Hydroquinidine, Ibutilide, Indecainide, Indenolol, Ipratropium Bromide, Landiolol, Lidocaine, Lorajmine, Lorcainide, Meobentine, Mexiletine, Moricizine, Nadoxolol, Nifenalol, Oxprenolol, Penbutolol, Pentisomide, Pilsicainide, Pindolol, Pirmenol, Practolol, Prajmaline, Procainamide Hydrochloride, Pronethalol, Propafenone, Propranolol, Pyrinoline, Quinidine, Sematilide, Sotalol, Talinolol, Tedisamil, Tilisolol, Timolol, Tocainide, Verapamil, Xibenolol.

Other biologically active molecules can also be incorporated in the delivery system. These biologically active molecules can include proteins, peptides, lipids, oligonucleotides, DNA, RNA, carbohydrates and imaging agents.

Proteins and peptides which may be used in accordance with the present invention include enzymes such as proteases (e.g. bromelain, papain, collagenase, elastase), lipases (e.g. phospholipase C), esterases, glucosidases, hyaluronidase, exfoliating enzymes; antibodies and antibody derived actives, such monoclonal antibodies, polyclonal antibodies, single chain antibodies and the like; reductases; oxidases; peptide hormones; natural structural skin proteins, such as elastin, collagen, reticulin and the like; anti-oxidants such as superoxide dismutase, catalase and glutathione; free-radical scavenging proteins; DNA-repair enzymes, for example T4 endonuclease 5 and P53; antimicrobial peptides, such as magainin and cecropin; a milk protein; a silk protein or peptide; and any active fragments, derivatives of these proteins and peptides; and mixtures thereof an anti-viral agent (such as acyclovir); an anti-hemorrhoid compound, an anti-wart agent (such as podophyllotoxin) and a plant extract and mixtures thereof.

Cytokines can also be incorporated into the delivery system. The cytokines include vascular endothelial growth factor (VEGF), endothelial cell growth factor (ECGF), fibroblast growth factor (FGF), insulin-like growth factor (IGF), bone morphogenic growth factor (BMP), platelet-derived growth factor (PDGF), epidermal growth factor (EGF), thrombopoietin (TPO), interleukins (IL1-IL15), interferons (IFN), erythropoietin (EPO), ciliary neurotrophic factor (CNTF), colony stimulating factors (G-CSF, M-CSF, GM-CSF), glial cell-derived neurotrophic factor (GDNF), leukemia inhibitory factor (LIF), and macrophage inflammatory proteins (MIP-1a,-1b,-2).

Genetic material can also be incorporated in the delivery system. Gene therapy can be used to introduce an exogenous gene in an animal to supplement or replace a defective or missing gene. For example, genes including but not limited to, genes encoding for HLA-B, insulin, adenosine deaminase, cytokines and coagulant factor VIII can be incorporated into the matrix and released over a fixed time period. The desired material can be operably linked to a variety of promoters well known in the art. Examples of promoters include, but are not limited to, an endogenous adenovirus promoter, such as the E1 a promoter or the Ad2 major late promoter (MLP) or a heterologous eucaryotic promoter, for example a phosphoglycerate kinase (PGK) promoter or a cytomegalovirus (CMV) promoter. Similarly, those of ordinary skill in the art can construct adenoviral vectors using endogenous or heterologous poly A addition signals.

The delivery system can be prepared in the form of a thin slab or film, microparticles or nanoparticles, and gels. The delivery system can also be in the form of a coating or part of a stent or catheter, a vascular graft or other prosthetic device.

The delivery system can be formed by solvent casting, emulsion solvent evaporation, electrospinning and other methods known to those skilled in the art.

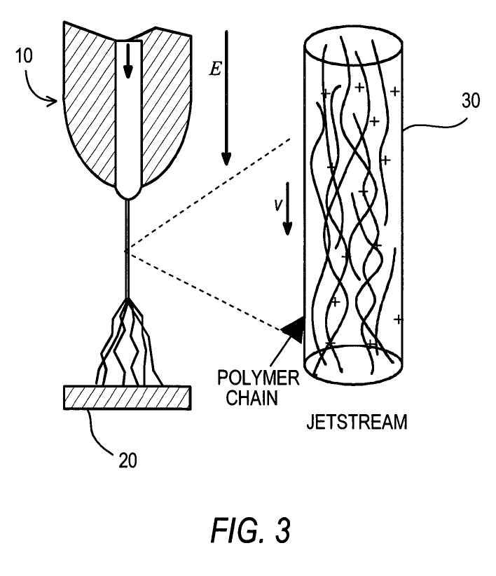

In electrospinning fibers are obtained from a solution using electricity. A schematic illustration of electrospinning is shown in FIG. 3. A solution is introduced into a spinneret 10. Voltage is applied to the spinneret to discharge the solution which flows to a target ground 20 and is spun into microfibers or polymer chains 30.

In the following examples, except for Example 1, the PEG content of the PEG-g-chitosan was 5.1 mole %. The molecular weight of the PEG used was 2,000. Poly(lactide-co-glycolide) 75:25 (PLGA, MW 130,000) was supplied by Birmingham Polymers, Inc. (Birmingham, USA). Methoxy poly(ethylene glycol) (Me-PEG, MW 2 000) and chitosan (85% deacetylation degree) were purchased from Sigma (St. Louis, USA). Phthalyl anhydride, trimethylphenyl chloride, dimethylaminopyridine (DMAP), N-hydroxy succinimide (HOSC), dicyclohexylcarboimide (DCC) and 1,1′-carbonyldiimidazole (CDI) were obtained from Aldrich (Milwaukee, USA). Dimethylformamide (DMF) and pyridine were dried with 4 Å molecular sieves prior to use. Tetrahydrofuran (THF) was dehydrated with CaH₂. All other solvents were used as received. All other chemicals were of reagent grade and distilled and deionized water was used. ¹H NMR spectra were obtained on a DMX500 NMR Spectrometer (Brucker) at room temperature using DMSO-d6 or CDCl₃ as solvent and Me₄Si as internal reference. Morphology of the electrospun films was observed on a JEOL JSM-5300 scanning electron microscopy (SEM). Samples for SEM were dried under vacuum, mounted on metal stubs, and sputter-coated with gold-palladium for 30 to 60 seconds.

### EXAMPLE 1

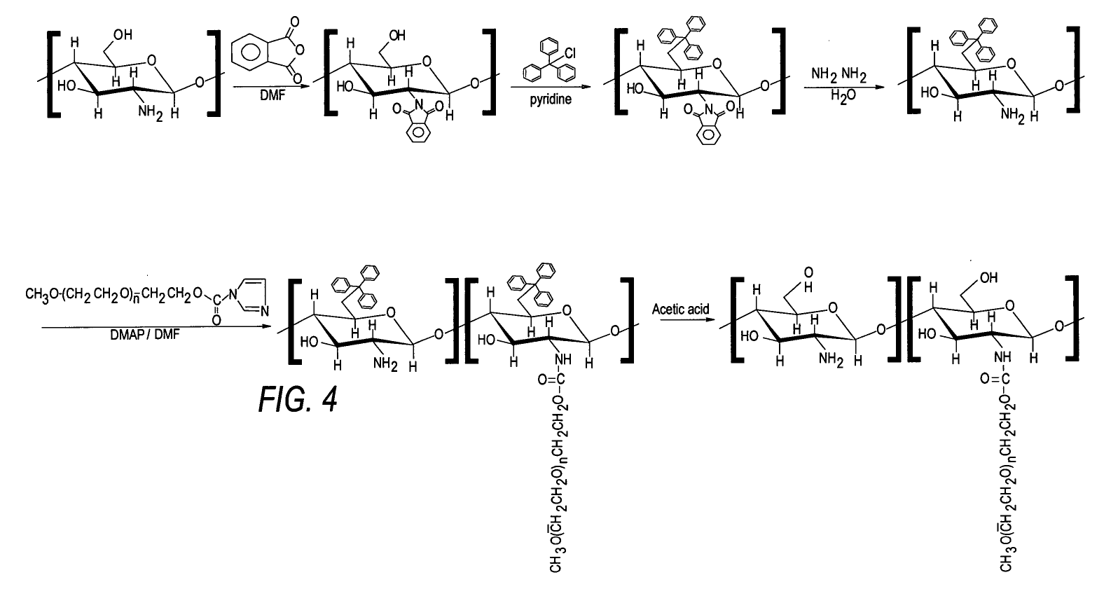

A synthetic process for making PEG-g-chitosan in accordance with Nishimura S., Kohgo O. and Kurita K.; Chemospecific manipulations of a rigid polysaccharide: syntheses of novel chitosan derivatives with excellent solubility in common organic solvents by regioselective chemical modifications; Macromolecules 1991; 24: 4745-4748 is illustrated in FIG. 4. CHN (chitosan) catalog number C3646 obtained from Sigma is modified by phthalation of its amino groups, triphenylmethylation of its hydroxyl groups and subsequent deprotection of amino groups to generate CHN analogs soluble in organic solvents. The hydroxyl group at one end of methyl-PEG is activated with carbonyldiimidazole (CDI), and is conjugated to CHN by using dimethylaminopyridiene as a catalyst. The PEG-g-triphenylmethyl-CHN formed is deprotected to give PEG-g-CHN. The unreacted PEG is removed by dialysis (MW cutoff 10,000). PEG content in the co-polymer can be adjusted by changing the [activated PEG]: [triphenylmethyl-CHN] feed ratio. Using this synthetic scheme, the graft level of PEG to CHN can reach as high as 50%. The PEG-g-Chitosan obtained in this manner is soluble in both organic solvent and water. The solubility is dependent upon the amount of PEG grafted onto the amino groups of native chitosan as shown in Table 2 and Table 3.

TABLE 2

| PEG/—NH₂ ratio | PEG graft level | | Yield | Solubility | | |
|---|---|---|---|---|---|---|
| (mole/mole) | Mole % | Weight % | (%) | DMF | H₂O | CHCl₃ |
| 0.04 | 1.8 | 14.5 | 72 | − | − | − |
| 0.15 | 5.1 | 41.1 | 67 | + | − | − |
| 0.8 | 20.6 | 165.6 | 54 | + | + | − |
| 4.0 | 48.7 | 392.5 | 51 | + | + | + |

TABLE 3
Solubility of PEG-g-Chitosan

| PEG/—NH₂ | |
|---|---|
| (mole %) | Solvent |
| 5.2 | DMF |
| | DMSO |
| 24.2 | DMF |
| | DMSO |
| | Water |
| 45.2 | DMF |
| | DMSO |
| | Water |
| | Chloroform |

### EXAMPLE 2

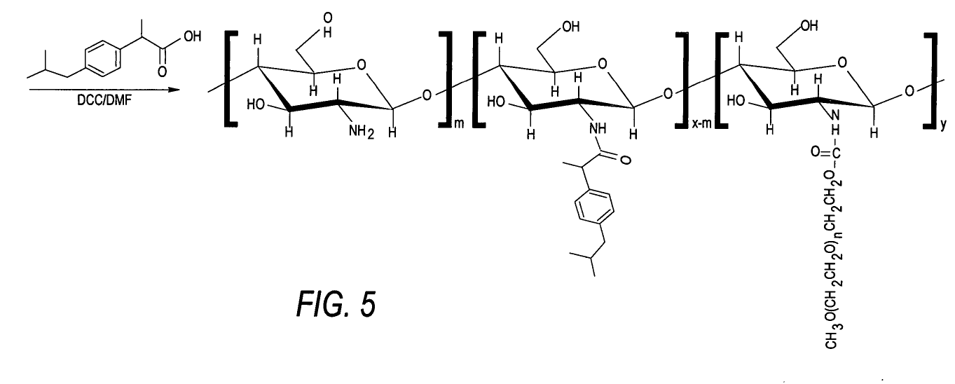

Ibuprofen can be conjugated to PEG-g-CHN as illustrated in FIG. 5. Ibuprofen was activated by reacting with HOSC and DCC (in equal molar ratio) in dried DMF for 1 day. The precipitates were filtered and PEG-g-CHN solution in dried DMF was added to the filtrate. The mixture was kept at room temperature under dry nitrogen atmosphere with constant stirring for 3 days. The conjugate was obtained by pouring the mixture into ethyl ether. The ibuprofen graft level of PEG-g-CHN-ibuprofen was determined by ¹H NMR (DMX500, Brucker).

The Ibuprofen loading in the conjugates could be adjusted by changing the [Ibuprofen]:[PEG-g-CHN] feed ratio.

TABLE 4

| | |
|---|---|
| Ibuprofen/ | Ibuprofen loading |

| —NH₂ ratio | Mole % | Weight % |
|---|---|---|
| 0.1 | 8 | 5.77 |
| 0.3 | 24 | 15.5 |
| 0.5 | 45 | 25.61 |

The PEG-g-chitosan in Table 4 had a PEG graft level of 5.1 mole %.

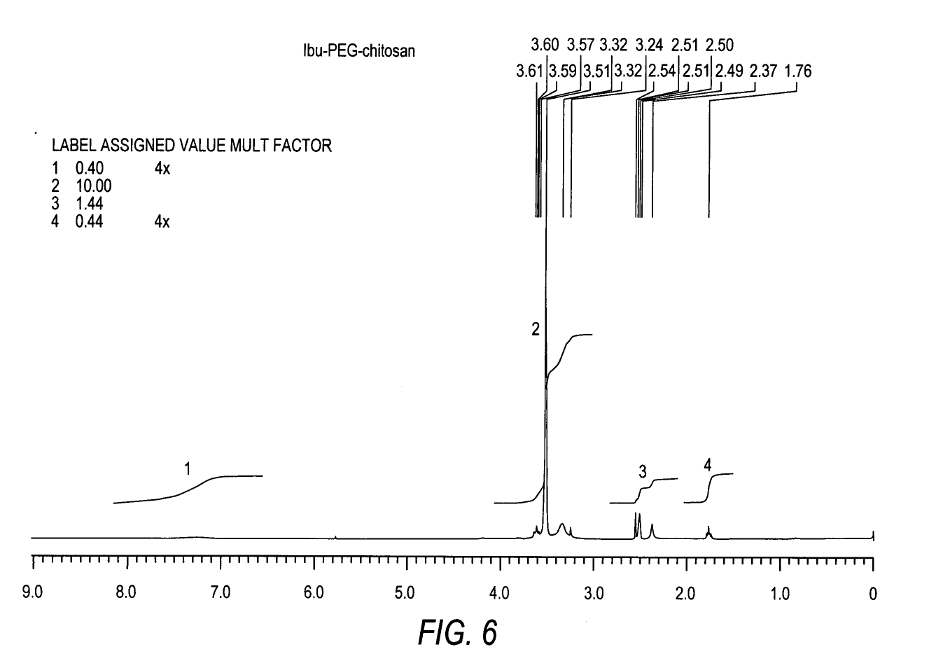

The structures of PEG-g-CHN-Ibuprofen co-polymers can be characterized by ¹H NMR as shown in FIG. 6, and their Ibuprofen loadings can be calculated according to the integral areas of the corresponding signals. The signals at 7.3 ppm are attributed to the aromatic protons of ibuprofen The signals at 3.51 ppm are attributed to the protons of PEG, and signals at 3.3 and 3.6 ppm are attributed to the protons of chitosan. IR spectroscopy was also used to characterize the PEG-g-CHN-Ibuprofen co-polymer, the presence of ether absorbance at 1108 cm⁻¹ in the IR spectrum (not shown) also indicates the grafting of PEG to chitosan.

### EXAMPLE 3

300 mg of PLGA, 200 mg of PEG-g-chitosan and 50 mg of ibuprofen are co-dissolved in 10 ml of dimethylformamide. The mixed solution is then poured into a Teflon Petri dish and left in a vacuum-evaporator at room temperature. It takes approximately one week to remove all the solvent (dimethylformamide). The film can then be detached from the Petri dish.

### EXAMPLE 4

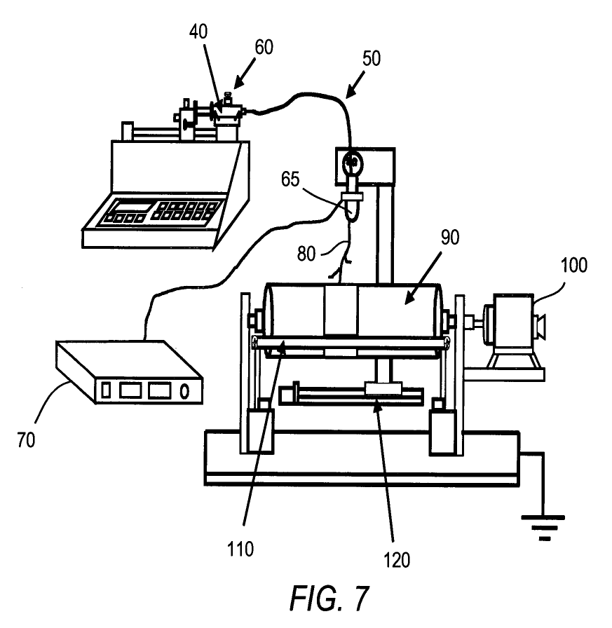

A multi-jet electrospinning instrument arrangement as shown in FIG. 7 can be used to prepare PEG-g-Chitosan/PLGA composite membrane. A polymer solution was prepared by dissolving 350 mg of PLGA and 350 mg of PEG-g-CHN in 1 ml of DMF. The mixture was thoroughly mixed overnight by vortexing. The polymer solution 40 was delivered to the exit hole of the electrode (spinneret 65 with a hole diameter of 0.7 mm) by a programmable pump 60 (Harvard Apparatus, MA). The flow rate range could be adjusted to 5-100 ml per minute. A positive high-voltage supply 70 (Glassman High Voltage Inc.) was used to maintain the voltage in a range of 0-30 kV. The collecting plate was placed on a rotating drum 90, which was grounded and controlled by a stepping motor 100. The distance of electric field was fixed at 150 mm. A heating rod 110 was installed to accelerate solvent evaporation. The films formed were placed in a vacuum oven at room temperature to fully eliminate solvent residuals.

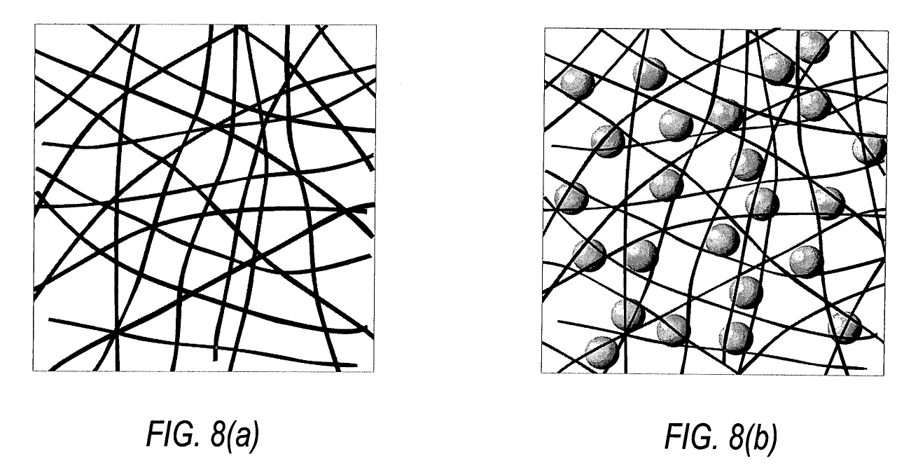

In comparison with the films prepared by the conventional solution casting solvent evaporation techniques, the films prepared by electrospinning have nanofibrous structure and were extremely porous with a high surface area-to-volume ratio. FIGS. 8A and 8B are two schematic illustrations of fiber mesh and micro/nanoparticles embedded in fiber mesh in accordance with the invention of the many potential configurations. The electrospun membranes are highly porous and their densities are approximately one-fifth of that of neat resin (or films prepared by conventional solution cast-solvent evaporation).

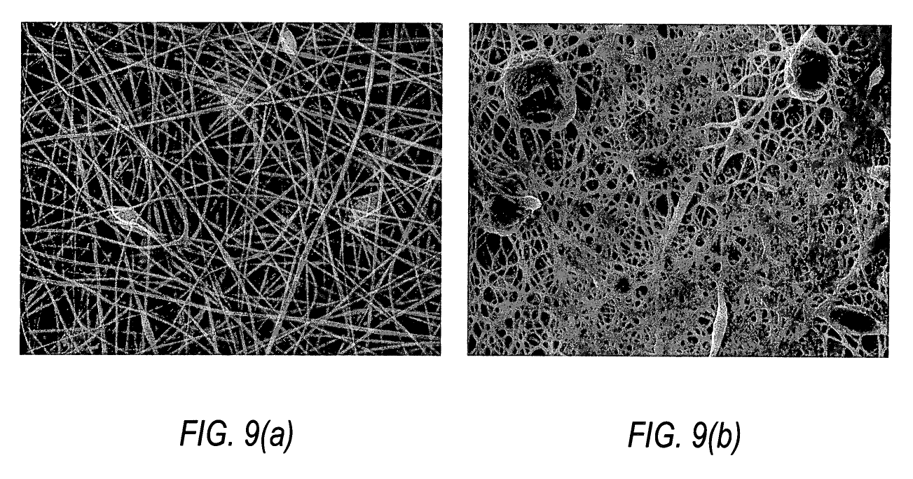

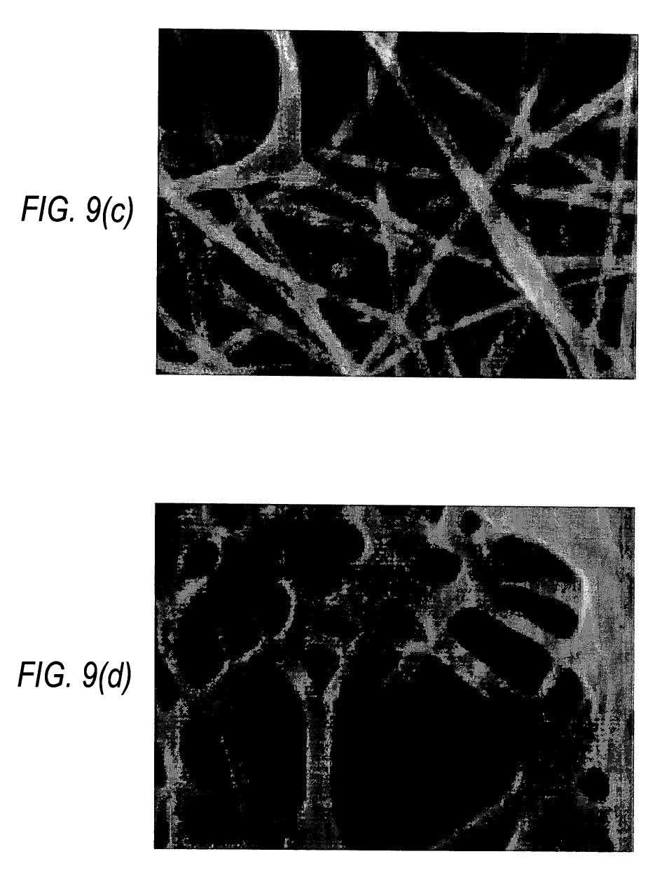

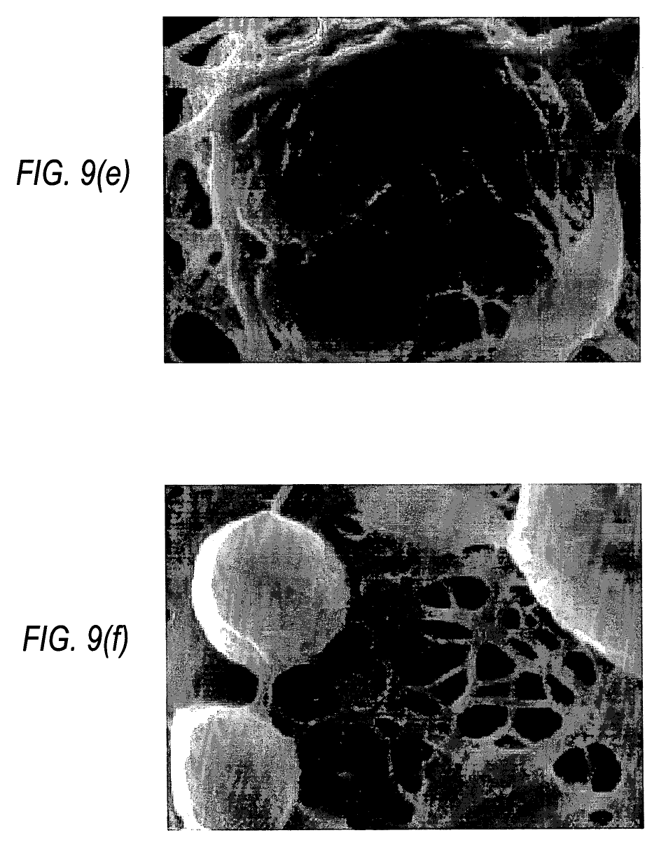

FIG. 9(a)-(f) are the SEM for composite films with different PLGA/PEG-g-CHN ratios. The morphology of the composite film (PLGA/PEG-g-CHN ratio at 90:10) was very similar to that of pure PLGA film mainly composed of nanofibers. With an increase in PEG-g-CHN content, to 80:20 as shown in FIG. 9(b) there appeared to be an increase in spherical structures. The sizes of these spherical structures were in the range of 2-10 μm. It has been reported in the literature that PEG-g-CHN could form aggregates in aqueous solution by hydrogen bonding. The size of PEG-g-CHN in DMF solution was measured by laser light scattering and found that the copolymer also associated with each other in DMF and the average size of the aggregates was 200 nm. The self-association characteristics rendered PEG-g-CHN difficult to be properly orientated even in the presence of electrostatic field (during the processing by electrospinning), leading to the instability of the liquid jets and occurrence of large amounts of spherical structures in the films prepared

### EXAMPLE 5

350 mg of PLGA (75:25, MW 138,000), 150 mg of PEG-g-Chitosan (PEG graft level 5.1%) and 25 mg of ibuprofen (5% loading) are dissolved in 10 ml of dimethylformamide (DMF) to form a feedstock. The mixture 40 is delivered through teflon tubing 50 by a syringe pump 60 to a spinneret 65 (hole diameter=0.7 mm) at a flow rate of up to 5 ml/minute. Up to 30 kV from a power supply 70 is applied to discharge the polymer feedstock, which eventually form microfibers 80. The microfibers are collected on a rotating drum 90, which is grounded and controlled by a stepping motor 100. A heating rod 110 is used to accelerate solvent evaporation. The recovered microfiber membrane is then placed in a vacuum oven at room temperature to fully eliminate the solvent. The spinneret 65 can be attached to a mobile base 120. Various nanostructured membranes can be prepared by electrospinning.

### EXAMPLE 6

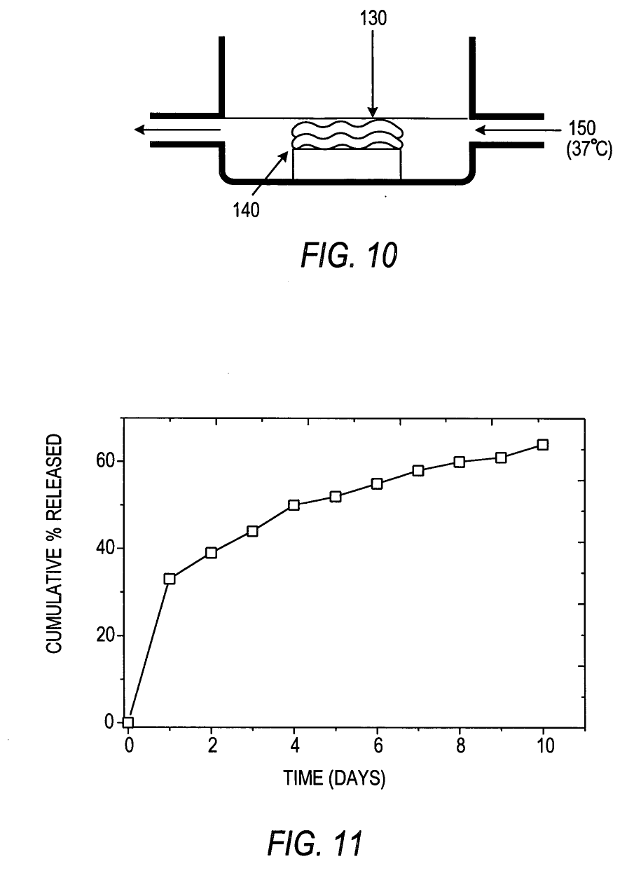

A PEG-g-CHN/PLGA cast film 130 with Ibuprofen dispersed in it (approximate dimension: 0.8×1.2 cm, 2% ibuprofen loading) was overlaid on a piece of human atrial tissue 140 (approximate dimension: 0.8×1.1 cm) in the configuration depicted in FIG. 10. Tyrode solution 150 was perfused through the bath to maintain the tissue. After 2 hours, the atrial tissue was removed, homogenized and extracted for ibuprofen. The ibuprofen concentration was determined by HPLC at ambient temperature [column: Phenomenex Synergi 4μ POLAR-RP 80 A, mobile phase: 20 mM KHPO₄/50% acetonitrile/50% water at pH 3.0, flow-rate: 1 ml/min at 1120 psi, detector: UV at 230 nm and 100 mV scale]. The amount of ibuprofen detected in the tissue was 1.12 μg/mg. tissue.

### EXAMPLE 7

The Ibuprofen release kinetics of a PEG-g-CHN/PLGA film (with 0.9% Ibuprofen loading) was evaluated. A sample of the PEG-g-CHN/PLGA/Ibuprofen film was incubated in a 0.1M pH 7.4 phosphate buffered saline (PBS) at 37° C. in a container under constant agitation. At stipulated time intervals, the PBS was withdrawn from the container and it was replenished with a fresh aliquot of PBS. The concentrations of Ibuprofen samples collected were determined by HPLC. The Ibuprofen release profile is depicted in FIG. 11.

### EXAMPLE 8

Ibuprofen release kinetics of various preparations of PLGA/PEG-g-CHN-Ibuprofen films were compared. Three types of electrospun polymer films containing ibuprofen were prepared; (i) pure PLGA films with 5% ibuprofen, (ii) PLGA/PEG-g-CHN (70/30 ratio) films with 5% ibuprofen, and (iii) PLGA/PEG-g-CHN-Ibuprofen films with 4.7% ibuprofen covalently conjugated. These films (approximately 20 mg per sample) were immersed in 2 ml of 0.1 M, pH 7.4 PBS incubated at 37° C. At pre-determined time-points, the liquid phases were withdrawn and replaced with fresh aliquots of PBS. The ibuprofen contents in the samples collected were determined by a UV-Vis spectrophotometer at 264 nm

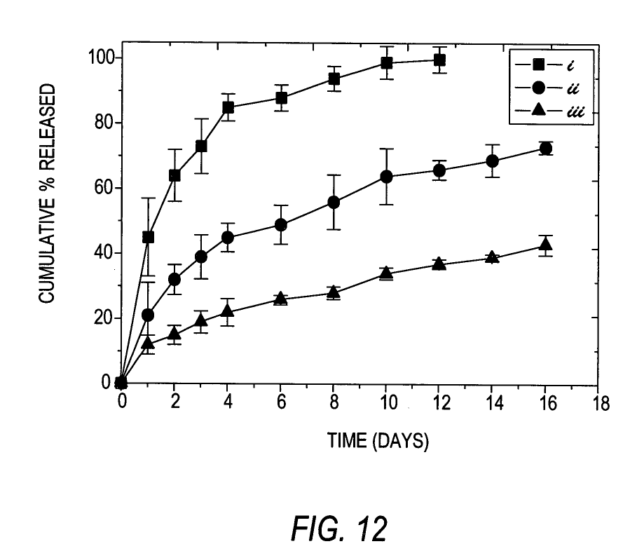

Ibuprofen release profiles from three kinds of the films are shown in FIG. 12. An initial burst release was observed from the electrospun PLGA film containing 5% ibuprofen. The drug release profile was likely due to the highly porous structure of the electrospun film and the lack of interaction between PLGA and ibuprofen. Consequently, more than 85% of the film ibuprofen content was released after 4 days. In contrast, the blending of PEG-g-CHN greatly moderated the release of ibuprofen incorporated into the electrospun PLGA/PEG-g-CHN film. The electrostatic interaction between the carboxyl moieties of ibuprofen molecules and the cationic chitosan could hinder the release of ibuprofen from the composite film, consequently, more moderated release kinetics were observed. The electrospun films prepared by blending PLGA with PEG-g-CHN-Ibuprofen where ibuprofen was covalently conjugated to the PEG-g-CHN showed a pseudo-linear release kinetics. Less than 40% of ibuprofen was released after 16 days.

### EXAMPLE 9

DNA-loaded composite microspheres can be prepared by a water-in-oil-in water emulsion solvent evaporation method. Briefly, 250 mg of PLGA is dissolved in 3 ml of chloroform and 50 mg of PEG-g-chitosan in 2 ml of dimethylsulfoxide (DMSO). The above two solutions are mixed. 1 ml of aqueous DNA solution (3 mg of DNA) is emulsified into the PLGA and PEG-g-chitosan mixture by a mechanical stirrer (at 2000 rpm) to form a water-in-oil emulsion. It is then poured into 25 ml of 5% polyvinyl alcohol (PVA) aqueous solution being stirred at 1000 rpm to form a water-in-oil-in-water emulsion. The complex emulsion is agitated with a magnetic stirrer overnight at room temperature to allow the organic solvents to evaporate. The microspheres can be collected by centrifugation.

### EXAMPLE 10

Protein (such as cytokines or other bioactive agents) or DNA loaded nanoparticles can be prepared by chelating protein or DNA to PEG-g-Chitosan/PLGA composite nanoparticles. Briefly, 250 mg of PLGA is dissolved in 3 ml of chloroform and 50 mg of PEG-g-chitosan in 2 ml of dimethylsulfoxide (DMSO). The above two solutions are mixed by rapid stirring, and then poured into 25 ml of 5% PVA aqueous solution. This mixture is then stirred at low speed (<500 rpm) for 5 minutes to form an oil-in-water emulsion. The emulsion is stirred with a magnetic stirrer overnight at room temperature to allow the organic solvents to evaporate. The nanoparticles can be collected by centrifugation followed by extensive washing and lyophilization. The PEG-g-Chitosan/PLGA nanoparticles are then dispersed in buffer and DNA (or protein solution) is added to it and allowed to incubate overnight. The DNA/PEG-g-Chitosan/PLGA (or Protein/PEG-g-Chitosan/PLGA) nanoparticles can be recovered by centrifugation.

### EXAMPLE 11

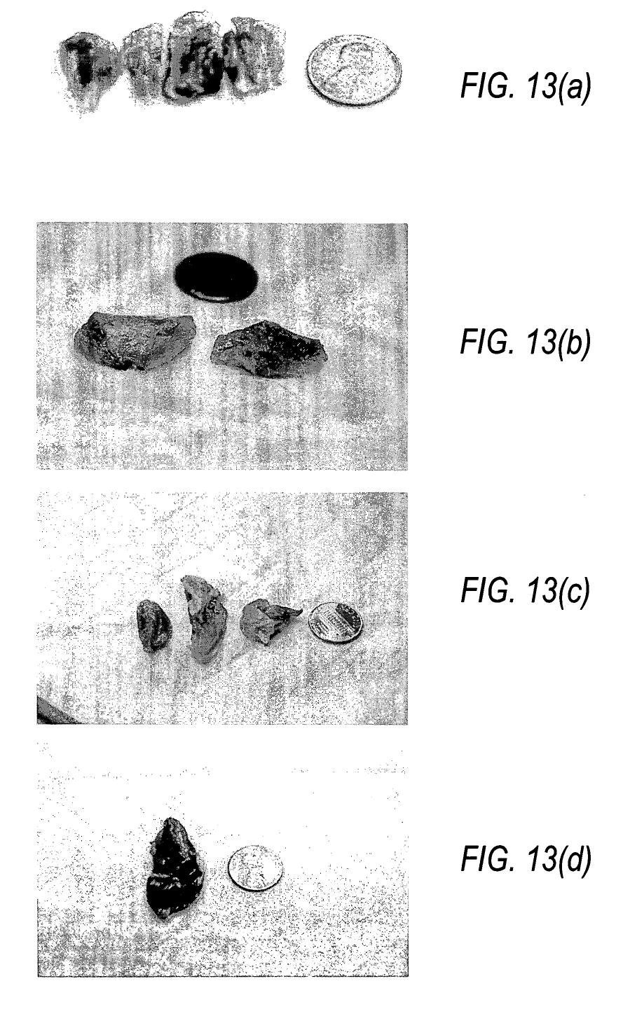

pCMVbeta beta-galactosidase plasmid DNA vector (with a cytomegalovirus promoter) was obtained from BD Bioscences Clontech, Palo Alto, Calif. 10 milligrams of DNA-polymer microspheres prepared in accordance with Example 10 were injected into rat hindlimb muscles (Sprague-Dawley, weighed 450-500 grams). The DNA content (loading) of the DNA-polymer microspheres were 1%. The animals were sacrificed at 1, 3, 6 and 12 weeks. Two animals per group were injected. The hindlimb muscles were retrieved and fixed in 10% buffered formalin solution for 3 to 5 days. Thereafter, each hindlimb muscle was incubated in 20 ml of X-Gal reagent solution following published procedure at 37° C. overnight. An animal was used as negative control, i.e. not injected with anything and X-Gal staining showed up negative. Photographs of the hindlimb muscles are shown in FIG. 13A-13D. As can be seen from the blue staining in FIG. 13 beta-galactosidase DNA was incorporated into the rat DNA using the delivery system of the invention.

Other polymers that are soluble in organic solvents can be blended with PEG-g-Chitosan to prepare microspheres or nanoparticles using similar procedures described above.

The films/membranes of the invention can be used for drug delivery. In addition, by conjugating the proper mix of cytokines, they can also be used for tissue engineering. The microspheres can be used for drug delivery, gene therapy, tissue engineering and diagnostics.

Fibronectin or fibroblast growth factor can be conjugated to a biodegradable PEG-g-Chitosan/PLGA electrospun aneurysm coil. This biodegradable and biocompatible aneurysm coil can be used to replace the platinum coils that are currently being used to treat inoperable aneurysm. Bone morphogenic protein or the DNA encoding bone morphogenic protein can be conjugated to a PEG-g-Chitosan/PLGA device as a vehicle for promoting bone fracture healing. Vascular Endothelial Growth Factor and/or Platelet Derived Growth Factor and/or angiopoetin (either as protein or DNA/or any combination) can be conjugated to PEG-g-Chitosan/PLGA microspheres or nanoparticles to promote angiogenesis and vasculogenesis. Cytokines (either as protein or DNA/or any combination) can be conjugated to PEG-g-Chitosan/PLGA electrospun membrane to promote chronic wound healing. PEG-g-Chitosan/PLGA electrospun membrane that does not shrink can be used to prevent post-operative tissue adhesion.

The above description is illustrative and not limiting. Further modifications will be apparent to one of ordinary skill in the art in light of the disclosure and appended claims.

We claim:

1. A composition comprising PEG-g-chitosan and a water insoluble polymer selected from the group consisting of polylactic acid, poly-lactide glycolide, polycaprolactones, ethylene vinyl acetate, polyanhydride and mixtures thereof.

2. A composition according to claim 1 further comprising a pharmaceutical.

3. A composition according to claim 1 further comprising one selected from the group consisting of RNA and DNA.

4. A composition according to claim 1 wherein the water insoluble polymer is biodegradable.

5. A composition according to claim 2 further comprising an organic solvent.

6. A composition according to claim 5 wherein the organic solvent is selected from the group consisting of dimethylformamide, dimethylsulfoxide and chloroform.

7. A delivery system prepared by the process comprising mixing PEG-g-chitosan and water insoluble polymer in an organic solvent, and separating the solvent to obtain the delivery system.

8. A delivery system prepared by the process comprising mixing PEG-g-chitosan, water insoluble polymer, and a pharmaceutical in an organic solvent, and separating the solvent to obtain the delivery system.

9. A delivery system prepared by the process comprising mixing PEG-g-chitosan, water insoluble polymer, and one selected from the group consisting of DNA and RNA in an organic solvent, and separating the solvent to obtain the delivery system

10. A method of making a time release pharmaceutical delivery system comprising:
mixing PEG-g-chitosan, a pharmaceutical and water insoluble polymer in an organic solvent, and
separating the solvent to obtain the delivery system.

11. A method according to claim 10 wherein the water insoluble polymer is poly-lactide glycolide.

12. A method according to claim 10 wherein the pharmaceutical is ibuprofen.

13. A method of delivering a pharmaceutical agent to a tissue comprising:
mixing PEG-g-chitosan, water insoluble polymer and a pharmaceutical agent in an organic solvent,
separating the solvent to obtain a delivery system, and
contacting the tissue with the delivery system.

14. A method according to claim 13 wherein the tissue is cardiac tissue.

15. A method according to claim 13 wherein the pharmaceutical agent is at least one selected from the group consisting of an anti-inflammatory, an antimicrobial, an antiviral, an antifungal and an antitumor agent.

16. A method of delivering DNA to a tissue comprising:
mixing PEG-g-chitosan, water insoluble polymer and DNA in an organic solvent,
separating the solvent to obtain a delivery system, and
contacting the tissue with the delivery system.

17. A method of delivering a biologically active molecule to a tissue comprising:
mixing PEG-g-chitosan, water insoluble polymer and at least one biologically active molecule in an organic solvent,
separating the solvent to obtain a delivery system, and
contacting the tissue with the delivery system

18. A method of reducing the inflammatory response of a tissue comprising:
mixing PEG-g-chitosan, water insoluble polymer and at least one anti-inflammatory agent in an organic solvent,
separating the solvent to obtain a delivery system, and
contacting the tissue with the delivery system.

19. A method according to claim 18 wherein the anti inflammatory is ibuprofen.

20. A method according to claim 18 wherein the tissue is cardiac tissue.

21. A method according to claim 18 wherein the tissue is atrial tissue.

22. A method of preventing post operative atrial fibrillation comprising:
mixing PEG-g-chitosan, water insoluble polymer and an anti-inflammatory in an organic solvent, the water soluble polymer being at least one selected from the group consisting of polylactic acid, poly-lactide glycolide, polycaprolactones, ethylene vinyl acetate and polyanhydride,
separating the solvent to obtain a delivery system, and
contacting cardiac tissue with the delivery system.
